(19) United States
(12) Patent Application Publication
(10) Pub. No.: US 2003/0153959 A1
(43) Pub. Date: Aug. 14, 2003

(54) Neural stimulation system providing auto adjustment of stimulus output as a function of sensed coupling efficiency

(76) Inventors: James R. Thacker, Eureka, MO (US); Carla Mann Woods, Los Angeles, CA (US)

Correspondence Address: ADVANCED BIONICS CORPORATION, 12740 SAN FERNANDO ROAD, SYLMAR, CA, 91342, US

(21) Appl. No.: 10/364,437

(22) Filed: Feb. 11, 2003

Related U.S. Application Data
(60) Provisional application No. 60/357,010, filed on Feb. 12, 2002.

(51) Int. Cl. A61N 1/18

(52) U.S. Cl. 607/48

(57) ABSTRACT

A neural stimulation system automatically corrects or adjusts the stimulus magnitude in order to maintain a comfortable and effective stimulation therapy. Auto correction of the stimulus magnitude is linked to the measurement of coupling efficiency. Because the events that lead to the necessity of an output amplitude change are all associated with how much electrical energy is coupled to the neural tissue, and because there are several physiologic parameters that reflect in some measure how much energy is actually coupled to the tissue, the measurement of the one or more of such physiologic parameters is used as an indicator of the electrode's effectiveness in providing therapeutic stimulation. The physiologic parameters that may be measured, and used by the invention as a measure of coupling efficiency, include: action potential(s); the optical transmissive and/or reflective properties of the tissue and fluids surrounding or adjacent the target neural tissue; the chemistry of the fluids and tissue near the electrodes, e.g., in the epidural space; or the changes in pressure that occur near the electrodes, e.g., in the epidural space. The relative change in the measured physiologic parameter is used as an indicator of the obstruction to current flow between the electrodes and the neural tissue. Such knowledge with respect to time thus permits the neural stimulation system to effectively auto-correct the output amplitude, thereby minimizing the occurrence of over-stimulation or under-stimulation.

This application claims the benefit of U.S. application Ser. No. 60/357,010, filed Feb. 12, 2002, which application is incorporated herein by reference.

### BACKGROUND OF THE INVENTION

The present invention relates to neural stimulation systems, such as a spinal cord stimulation (SCS) system, and more particularly to an output control system used with such neural systems that automatically maintains the output of the stimulation system at a comfortable and efficacious level.

By way of example, a spinal cord stimulation system, which represents one type of neural stimulation system with which the present invention may be used, treats chronic pain by providing electrical stimulation pulses through the electrodes of an electrode array placed epidurally near a patient's spine.

Spinal cord stimulation is a well accepted clinical method for reducing pain in certain populations of patients. SCS systems typically include an Implantable Pulse Generator (IPG) coupled to an array of electrodes at or near the distal end of an electrode lead. An electrode lead extension may also be used, if needed. The IPG generates electrical pulses that are delivered to neural tissue, e.g., the dorsal column fibers within the spinal cord, through the electrodes of the electrode array. In an SCS system, for example, the electrodes are implanted proximal to the dura mater of the spinal cord. Individual electrode contacts (the “electrodes”) may be arranged in a desired pattern and spacing in order to create an electrode array. Individual wires, or electrode leads, connect with each electrode in the array. The electrode leads exit the spinal cord and attach to the IPG, either directly, or through one or more electrode lead extensions. The electrode lead extension, in turn, when used, is typically tunneled around the torso of the patient to a subcutaneous pocket where the IPG is implanted.

The electrical pulses generated by the SCS stimulation system, or other neural system, are also referred to as “stimulation pulses”. In an SCS system, the stimulation pulses typically have the effect of producing a tingling sensation, also known as a paresthesia. The paresthesia helps block the chronic pain felt by the patient. The amplitude, or magnitude, of the stimulation pulses affects the intensity of the paresthesia felt by the patient. In general, it is desirable to have the paresthesia at a comfortable level, i.e., not too weak (because then the pain is not blocked), and not too strong (because the paresthesia can itself become painful and uncomfortable).

SCS and other stimulation systems are known in the art. For example, an implantable electronic stimulator is disclosed in U.S. Pat. No. 3,646,940 that provides timed sequenced electrical impulses to a plurality of electrodes. As another example, U.S. Pat. No. 3,724,467, teaches an electrode implant for neuro-stimulation of the spinal cord. A relatively thin and flexible strip of biocompatible material is provided as a carrier on which a plurality of electrodes are formed. The electrodes are connected by a conductor, e.g., a lead body, to an RF receiver, which is also implanted, and which is controlled by an external controller.

Representative techniques known in the art for providing for the automatic adjustment of stimulation parameters of an implantable stimulator are disclosed, e.g., in U.S. Pat. Nos. 5,913,882; 5,895,416; 5,814,092; 5,735,887; 5,702,429 and 4,735,204.

Patients having an SCS system have heretofore had to manually adjust the amplitude of the stimulation pulses produced by their SCS system in order to maintain the paresthesia at a comfortable level. This is necessary for a number of reasons. For example, postural changes, lead array movement (acute and/or chronic), and scare tissue maturation, all affect the intensity of the paresthesia felt by the patient. Because of these changes, i.e., because of postural changes, lead array movement, and scare tissue maturation, as well as other changes that may occur in the patient, the paresthesia can be lost, or can be converted to painful over-stimulation, thereby forcing the patient to manually adjust the output. There is a need for a method or system that would eliminate, or at least mitigate, the need to perform such manual adjustments. Such method or system would be of great benefit to the patient.

### SUMMARY OF THE INVENTION

The present invention addresses the above and other needs by providing a neural stimulation system that automatically corrects or adjusts the stimulus amplitude in order to maintain a comfortable and effective stimulation therapy. Auto correction of the stimulus amplitude is linked to a sensor that senses coupling efficiency. In one embodiment, coupling efficiency is determined by the measurement of action potentials. Because the events that lead to the necessity of an output amplitude change are all associated with how much electrical energy is coupled to the neural tissue, and because the action potentials resulting from stimulated tissue reflect how much energy is actually coupled to the tissue, i.e., the coupling efficiency, the measurement of the action potential(s) may be used as an indicator of the electrode's effectiveness in providing therapeutic stimulation. Other indicators of coupling efficiency may also be used. Thus, as an event, such as a postural change, lead array movement, or scare tissue maturation, occurs that allows more energy to couple from the emitting electrode or electrodes to the neural tissue, the more likely the stimulus will be painful, i.e., the more likely over-stimulation will occur. Conversely, as an event occurs that attenuates the coupled energy, the more likely the stimulus will not be sufficient to evoke the desired therapeutic effect. (under stimulation). In one embodiment, the relative change in the measured action potential is used as an indicator on the obstruction to current flow between the electrodes and the neural tissue. The knowledge of the action potential associated with an emitting electrode, or the action potential from neighboring electrodes, with respect to time permits the system to effectively auto correct the output amplitude, thereby minimizing the occurrence of over-stimulation or under-stimulation. In other embodiments, other means are used to determine the coupling efficiency.

One application for the present invention is for a Spinal Cord Stimulation (SCS) system wherein the invention provides a system that automatically corrects or adjusts the stimulation amplitude in order to maintain a comfortable and effective paresthesia. As postural change, lead array movement, scare tissue maturation, and the like occur, allowing more energy to couple from the emitting electrode or electrodes of the SCS system to the neural tissue, the more likely over-stimulation occurs. Conversely, as events occur that attenuate the coupled energy, the more likely a desired paresthesia does not occur (under stimulation). The relative change in the coupling efficiency provides a measure of the obstruction to current flow between the electrodes and the neural tissue. Coupling efficiency may be determined in various ways, e.g., measuring the action potential, measuring the impedance between electrodes, measuring any change in the electrode position, measuring changes in the optical properties of tissue surrounding the tissue, and the like. For example, the knowledge of the action potential associated with an emitting electrode, or the action potential from neighboring electrodes, with respect to time allows the system to effectively auto correct the output amplitude, thereby minimizing the occurrence of over-stimulation (excessive-threshold paresthesia) or under-stimulation (sub-threshold paresthesia).

In accordance with one aspect of the invention, there is provided a neural stimulation system having means for measuring the coupling efficiency that occurs at selected electrodes of an electrode array resulting from the application of electrical stimulating pulses to other selected electrodes of the electrode array. The amplitude and/or morphology of the measured coupling efficiency determined, e.g, by measuring action potentials, impedance, position, optical properties of surrounding tissue, and the like, are monitored over time for changes of significance, i.e., changes indicating that over-stimulation or under-stimulation is likely to occur. In response to such changes, adjustments are automatically made to the amplitude of the stimulus current so as to prevent painful over-stimulation or sub-threshold under-stimulation from occurring.

By way of example, as tissue healing (scar maturation) occurs following insertion of the electrode array, the measured coupling efficiency decreases, requiring a higher stimulation pulse amplitude to achieve the same therapeutic result. The time frame is usually a slowly changing morphology of the measured coupling efficiency, and a decrease in the maximum voltage associated with the coupling efficiency measurement. Such conditions, when detected, indicate that the stimulus output (amplitude of the stimulation pulse) should be gradually increased in order to compensate for the scar maturation effects.

By way of another example, a rapidly changing coupling efficiency usually indicates an acute movement of the electrode contact(s) of the electrode array relative to their proximity to the dura mater. Such rapid change could be due to a postural change or due to the electrode array permanently moving its location with respect to its former location in the epidural space. Whatever the cause, the change in the coupling efficiency in a relatively short period of time is indicative of a changing electrode environment, and this knowledge in turn suggests the corresponding necessity to change the amplitude of the output stimulus to compensate for such changes.

It is thus a feature of the present invention to provide a neural stimulation system wherein the output stimulus amplitude is automatically corrected or adjusted in order to compensate for coupling variations in the electrode-to-neural-tissue interface that cause more or less energy to reach the neural tissue from the electrode. In one preferred embodiment, such sensed coupling variations are determined by measuring or monitoring changes in a sensed action potential. In other preferred embodiments, such sensed coupling variations are determined by measuring or monitoring changes in other physiologic parameters that vary as a function of coupling efficiency, e.g., the optical reflective and/or transmissive properties of tissue surrounding the electrodes, the electrode/tissue interface impedance, or the like.

It is a further feature of the invention to provide a method of neural stimulation that includes the measurement of suitable physiologic parameter(s) that are indicative of the coupling efficiency of the electrical stimulation current to the neural tissue, and automatically adjusting the magnitude of subsequent stimulating current pulses in order to compensate for variations in the measured coupling efficiency.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows a representative neural stimulation system of the type with which the present invention may be used;

FIG. 2 shows the stimulation system of FIG. 1 being used as a Spinal Cord Stimulation (SCS) system, with the electrode array inserted alongside the spinal cord in the epidural space, in close proximity to the dura mater;

FIG. 3A is a block diagram of a system that automatically adjusts the amplitude of the stimulus current applied to neural tissue in accordance with the present invention;

FIG. 3B illustrates one method for generating and sensing action potentials in accordance with the invention;

FIG. 4 is a block diagram of a representative Implantable Pulse Generator (IPG) that may be used to practice the present invention;

FIG. 5 is a timing waveform diagram that depicts various types of action potentials (AP1, AP2, AP3) that may be sensed as a function of time in response to a stimulus pulse (SP1); and

FIG. 6 is a flow chart that shows a method of practicing the invention in accordance with one embodiment thereof.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

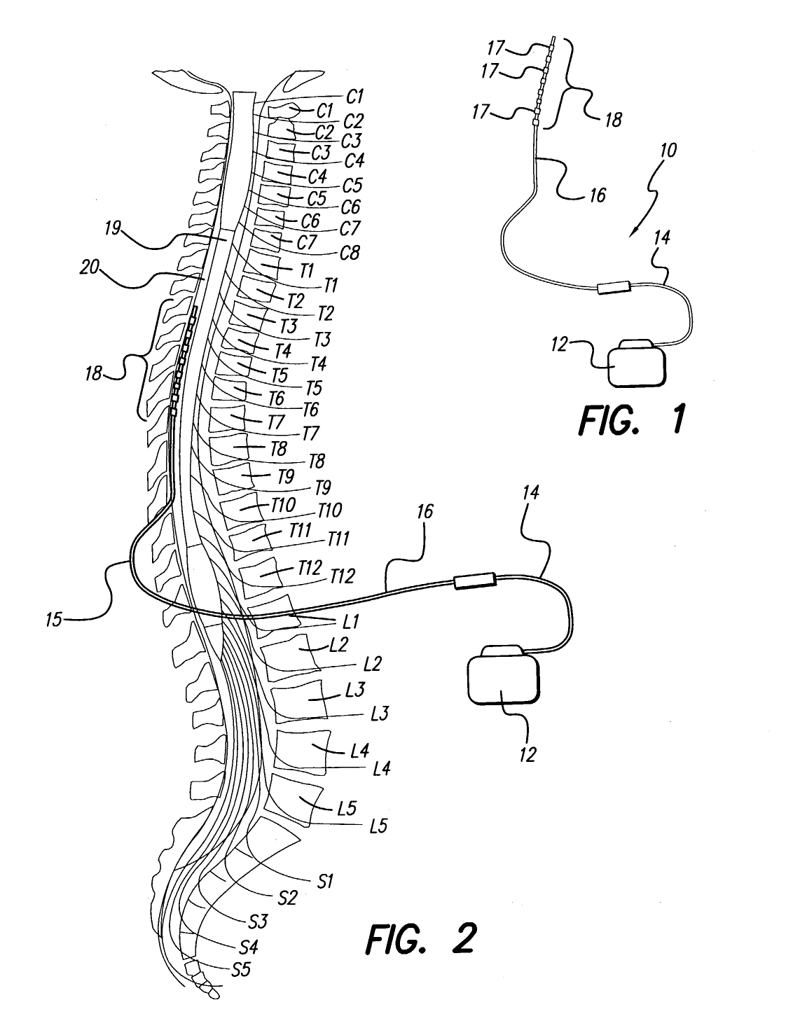

A representative neural stimulation system 10 is shown in FIG. 1. Such system typically comprises an Implantable Pulse Generator (IPG) 12, a lead extension 14, an electrode lead 16, and an electrode array 18. The electrode array includes a plurality of electrode contacts 17 (also referred to as “electrodes”). The electrodes 17 are arranged, for example, in an in-line array 18 near the distal end of the lead 16. Other electrode array configurations may also be used. The IPG 12 generates stimulation current pulses that are applied to selected ones of the electrodes 17 within the electrode array 18.

A proximal end of the lead extension 14 is removably connected to the IPG 12, and a distal end of the lead extension 14 is removably connected to a proximal end of the electrode lead 16, The electrode array 18, is formed on a distal end of the electrode lead 16. The in-series combination of the lead extension 14 and electrode lead 16, carry the stimulation current from the IPG 12 to electrodes of the electrode array 18. It should be noted that the lead extension 14 need not always be used with the neural stimulation system 10. The lead extension 14 is only needed when the physical distance between the IPG 12 and the electrode array 18 requires its use.

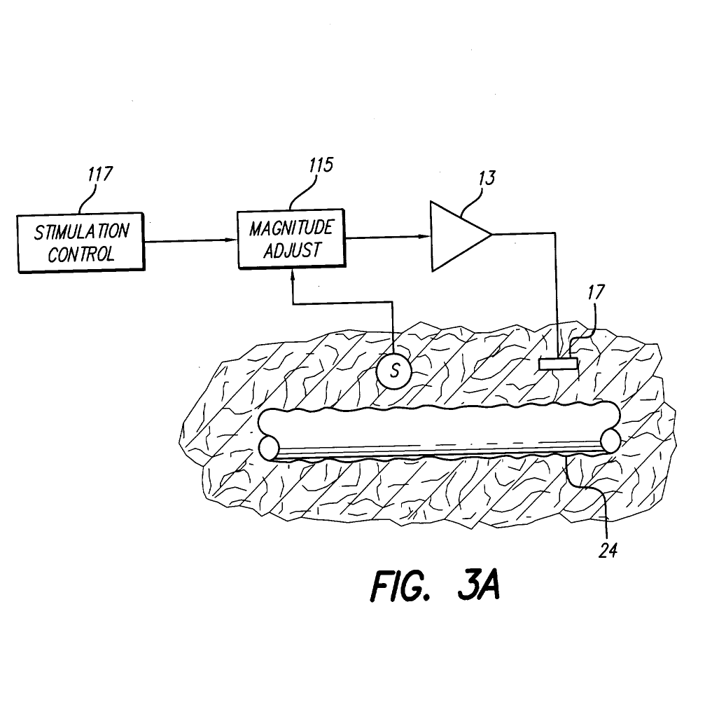

Turning next to FIG. 2, the neural stimulation system 10 is shown being used as a Spinal Cord Stimulator (SCS) system. In such configuration, the lead 16, and more particularly the electrode array 18, is implanted in the epidural space 20 of a patient so as to be in close proximity to the spinal cord 19. Due to the lack of space near the lead exit point 15 where the electrode lead 16 exits the spinal column, the IPG 12 is generally implanted in the abdomen or above the buttocks. The lead extension 14 facilitates locating the IPG 12 away from the lead exit point 15.

A more complete description of an SCS system may be found in U.S. Pat. No. 6,516,227, which patent is incorporated herein by reference in its entirety.

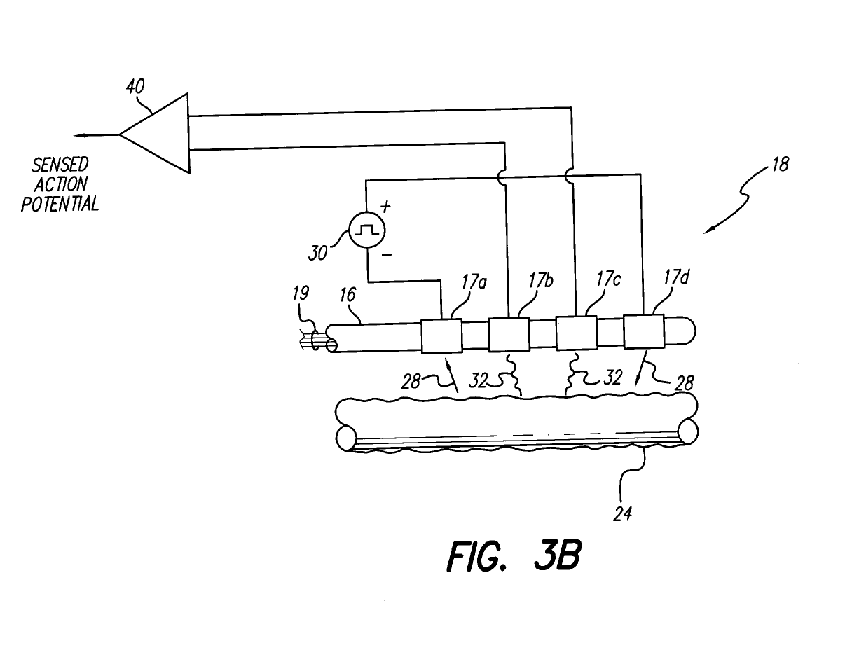

Next, with respect to FIG. 3A, there is shown a functional block diagram of a system that automatically adjusts the amplitude of the stimulus current applied to neural tissue in accordance with the present invention. As seen in FIG. 3A, an electrode 17 is placed in close proximity to neural tissue 24 that is to be stimulated. The electrode 17 is electrically connected to a current pulse generator 13 which generates a stimulus pulse having a magnitude that is set by magnitude adjust circuitry 115. The magnitude adjust circuitry 115 sets the magnitude of the stimulus pulse as specified by stimulation control circuitry 117. The stimulation control circuitry 117 usually comprises some sort of processor, or state logic, that operates in accordance with a stored program or state diagram. It initially sets the magnitude of the stimulus pulse to a programmed or predetermined value.

As the stimulus pulse is applied to the neural tissue 24, an appropriate sensor S senses the coupling efficiency between the stimulus current and the neural tissue. That is, the sensor S provides a measure of how effective the applied stimulus is at stimulating the neural tissue 24. The sensor S is connected to the magnitude adjust circuitry 115 so as to provide a feedback signal that indicates whether the magnitude of the stimulus needs to be adjusted up or down. For example, should the sensor S determine that very little of the energy is being coupled to the neural tissue 24, then the feedback signal provided through the sensor S automatically causes the magnitude adjust circuitry 115 to increase the magnitude of the stimulus pulse so that the total energy coupled to the neural tissue 24 remains about the same. Conversely, should the sensor S determine that more energy is being coupled to the neural tissue 24, then the feedback signal provided through the sensor S automatically causes the magnitude adjust circuitry 115 to decrease the magnitude of the stimulus pulse so that the total energy coupled to the neural tissue 24 remains about the same. Thus, it is seen that the magnitude adjust circuitry 115 automatically adjusts the magnitude, e.g., amplitude, of the stimulus pulse so that the energy coupled to the neural tissue remains more or less the same.

The sensor S may take various forms in accordance with the present invention. As described in more detail hereinafter, the sensor S may be, in one embodiment, a sensor that measures action potentials. In another embodiment, the sensor S may comprise optical circuitry that senses the optical properties of the body tissue surrounding or adjacent the target neural tissue that is stimulated. For example, the optical circuitry may comprise an optical transmitter and optical receiver that sense the reflective and transmissive properties of the body tissue surrounding the neural tissue. In accordance with this embodiment, optical energy of a prescribed frequency is emitted into the body tissue towards the target neural tissue 24 from an optical emitter included within the sensor S. Preferably this optical energy is pulsed, but in some embodiments, it may be continuous. An optical receiver is placed in the sensor S near the optical emitter so as to receive any optical energy of the same frequency that is reflected from the body tissue and/or neural tissue. The amount of optical energy received by the optical receiver will vary as a function of the reflective and transmissive properties of the tissue through which the optical energy passes, and from which the optical energy reflects. These optical properties in turn vary as a function of the coupling efficiency between the stimulus current and the neural tissue. Hence, a measure of these optical properties provides a measure of the coupling efficiency.

In still further embodiments, the sensor S may sense electrical impedance, conductance, or other electrical properties indicative of coupling efficiency.

In other embodiments, the sensor S may sense acoustical properties of the tissue surrounding the neural tissue 24, e.g., in order to determine the relative density, or amount of relative stress or pressure, within the tissue.

In yet additional embodiments, the sensor S may sense the chemical properties, e.g., pH, of the fluid in the epidural space.

In one preferred embodiment of the present invention, as has been indicated, coupling efficiency is determined by measuring action potentials resulting from a stimulus pulse being applied to the neural tissue. Thus, for purposes of such embodiment, the sensor S measures action potentials.

FIG. 3B shows one method for generating and sensing action potentials in accordance with this embodiment of the invention. Shown in FIG. 3B is a schematic representation of the distal end of the lead 16, including four electrode contacts 17a, 17b, 17c, and 17d. (It is to be understood that the lead 16 used with the neural stimulation system will have at least one electrode contact, and will usually have a plurality of electrode contacts, e.g., 4 or 8 or 16 or more electrode contacts. The four electrode contacts shown in FIG. 3B is intended to be illustrative only, and not limiting. It is also to be understood that the physical and electrical connection with the electrode contacts 17a, 17b, 17c and 17d is typically made through wires 19 that pass through the body of the lead 16. However, for simplicity of illustration in the schematic diagram of FIG. 3B, the electrical connection with the respective electrode contacts 17a, 17b, 17c and 17d is shown by wires external to the body of the lead 16.) The electrode contacts 17a, 17b, 17c and 17d are positioned in the epidural space near the dorsal column fibers 24 within or near the spinal cord.

In order to sense action potentials, stimulation pulses are applied from a pulse generator 30, to a selected pair of the electrode contacts, e.g., electrode contacts 17a and 17d. As connected in FIG. 3B, the polarity of the pulse generator 30 causes a current, represented by arrow 28, to be emitted from electrode contact 17d to the neural tissue 24. The current 28 flows through the nerve tissue 24 and surrounding tissue and returns to the pulse generator 30 through electrode contact 17a. The energy contained within the current 28 is coupled to the neural tissue 24 as a function of the coupling efficiency between electrode contacts 17a, 17d and the neural tissue 24. This coupling efficiency may vary, for many reasons, such as postural changes, relative movement between the lead 16 and tissue 24, or scare tissue maturation, to name just a few.

As the tissue 24 is excited, or subjected to the stimulation current 28, an action potential is created. Such action potential is schematically represented in FIG. 3B by the wavy lines 32. The action potential 32 may be sensed in various ways. One way is through electrode contacts 17b, 17c and a suitable pre-amplifier 40. The output signal obtained from the pre-amplifier 40 thus becomes a measure of the sensed action potential. Hence, by stimulating through one set of electrodes, 17a, 17d, and sensing through another set of electrodes, 17b and 17c, the action potential resulting from application of the current 28 to the nerve tissue 24 can be monitored.

It is to be emphasized that the technique shown in FIG. 3B for sensing an action potential is only representative of various ways that could be used. For example, with appropriate gating circuitry, the action potential could also be sensed through the same electrode contact(s) that are used to apply the stimulation pulse. This is possible because the action potential associated with most excited nerve tissue typically follows application of the stimulus current by a short delay, e.g., 0.1 to 3 milliseconds (msec). Thus, through the use of appropriate gating circuitry that switches the action potential monitoring circuitry on only after the stimulus pulse has been applied, the same electrode contacts can be used for both purposes (stimulating and monitoring).

Alternatively, separate, dedicated electrode contacts may be used to monitor the action potential. Such electrode contacts may be included on the lead 16, or may be included on a separate lead.

Further, it should be noted that one electrode contact associated with stimulation and/or monitoring may be included as part of the case of the IPG 12 (FIG. 1), e.g., as a ground or return electrode, in which instance both mono-polar stimulation and mono-polar sensing may be used. (In contrast, the stimulating and sensing configuration illustrated in FIG. 3B represents bipolar stimulation and bipolar sensing.) In a similar manner, multi-polar stimulation and multi-polar sensing could also be employed. (In this context, “multi-polar” refers to using multiple electrode contacts, i.e., three or more, for stimulating and/or sensing.)

Finally, it should be pointed out that other types of sensors may be employed, in addition to electrode contacts connected to a sense amplifier, in order to sense the action potential or to sense other physiologic parameters that provide a measure of the coupling efficiency between the stimulating electrodes and the neural tissue. For example, a pressure transducer could sense changes in pressure that occur in the epidural space, or impedance measurements could be made between the stimulating electrodes, or chemical sensors (e.g., a pH sensor) could measure changes in the chemistry of the fluids and tissue in the epidural space, or the like.

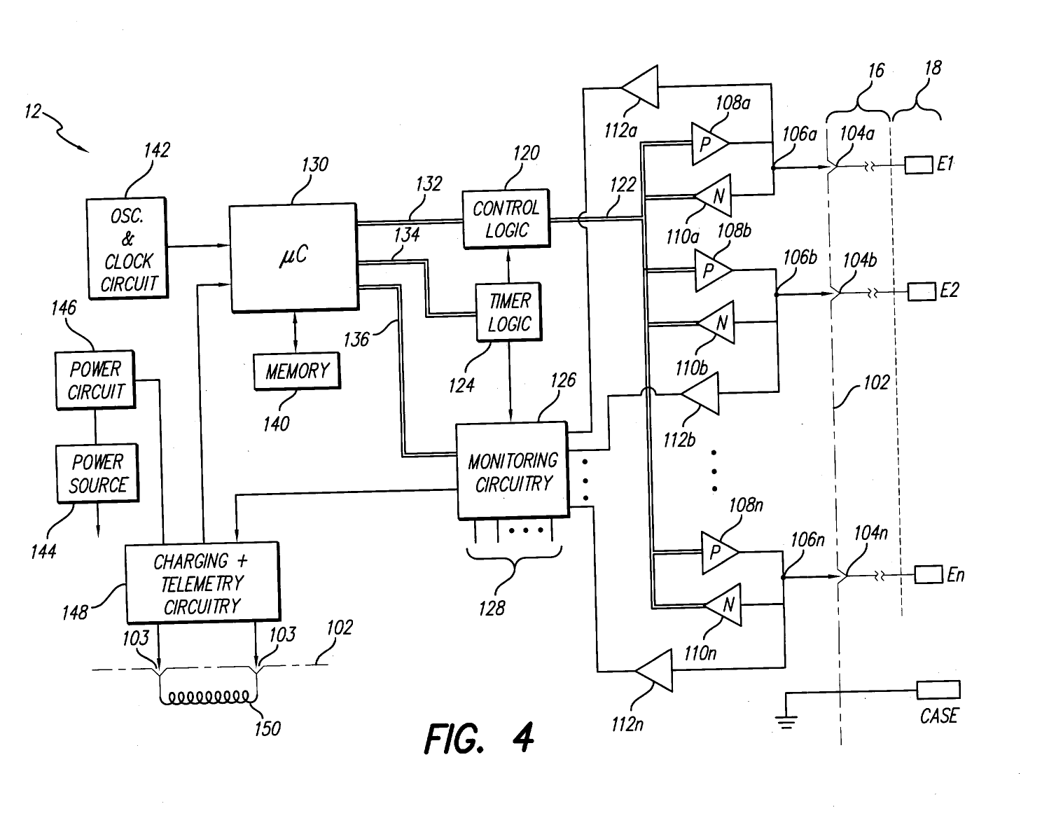

Turning next to FIG. 4, there is shown a functional block diagram of a representative Implantable Pulse Generator (IPG) 12 (or, with respect to FIG. 3, pulse generator 30) that may be used to practice the present invention. As seen in FIG. 4, the IPG 12 is connected to a multiplicity of electrode contacts E1, E2, . . . En, where n represents an integer of at least 3. The dotted-dashed line 102 in FIG. 4 represents the boundary between the outside of the IPG case (which is exposed to body tissues and fluids when the IPG is implanted) and the inside of the IPG case (which forms an hermetically sealed compartment wherein the electronic and other components are protected from the body tissues and fluids). Feed-through terminals 104a, 104b, . . . 104n are thus used to provide an electrical path through the IPG case wall 102. The feed-through terminals 104a, 104b, . . . are electrically connected to the electrodes E1, E2, . . . through wires within the lead 16.

Thus, it is seen that each electrode contact E1, E2, . . . En is connected through a respective feed-through terminal 104a, 104b, . . . 104n to a respective circuit node 106a, 106b, . . . 106n within the hermetically sealed IPG case. This node, in turn is connected to a P-DAC circuit 108a and an N-DAC circuit 110a, as well as a sense amplifier 112a. Each of the other circuit nodes 106b, . . . 106n within the IPG similarly have a respective P-DAC circuit, N-DAC circuit, and sense amplifier connected thereto.

A case electrode, CASE, may also be provided that effectively provides a common or return electrode that may be used with some stimulation and sensing configurations.

In operation, in order to generate a stimulus current pulse that is applied between electrodes E1 and E2, for example, the P-DAC circuit 108a, as controlled by control logic 120 over data bus 122, causes a stimulation current having a specified amplitude to be emitted from the node 106a, and hence to be emitted from the electrode contact El. At the same time, the N-DAC circuit 110B, similarly controlled by control logic 120, causes a stimulation current of the same magnitude to be received through node 106b, and hence through electrode contact E2. (Not shown in FIG. 4, but assumed to be present, are coupling capacitors connecting the respective nodes 106 and feed-through terminals 104.) In this way, a precisely controlled current is generated that flows from electrode contact E1 to electrode contact E2 through whatever body and nerve tissue resides between electrodes E1 and E2. The duration of the current flow, i.e., the width of the current pulse that is generated, is controlled by timer logic circuitry 124. The operation of this output circuitry, including alternative embodiments of suitable output circuitry for performing the same function of generating current stimulus pulses of a prescribed amplitude and width, is described more fully in the above-referenced U.S. Pat. No. 6,516,227.

Each sense amplifier 112a, 112b, . . . 112n connects its respective electrode node 106a, 106b, . . . 106n to monitoring circuitry 126. The monitoring circuitry 126 also monitors other signals 128 from various locations or components within the IPG, e.g., battery voltage, charge current, etc.

The control logic 120, the timer logic 124, and the monitoring circuit 126 are controlled or watched by a suitable micro-controller (μC) circuit 130. The μC circuit 130 is coupled to the control logic 120, the timer logic 124, and the monitoring circuitry 126 over data buses 132, 134 and 136, respectively.

Suitable memory circuitry 140 is likewise coupled to the μC 130, as is an oscillator and clock circuit 142. The μC 130, in combination with the memory circuit 140 and oscillator and clock circuit 142, thus comprise a microprocessor system that carries out a program function in accordance with a suitable program stored in the memory 140. Alternatively, for some applications, the function provided by the microprocessor system may be carried out by a suitable state machine.

Power for the IPG is provided by way of a suitable power source 144, such as a rechargeable battery. A power circuit 146 controls the charging or replenishment of the power source, as described more fully in the above-referenced U.S. Pat. No. 6,516,227.

The power circuit 146, the μC 130 and the monitoring circuitry 126 are also coupled to charging and telemetry circuitry 148. An antenna coil 150 is likewise coupled to the telemetry circuitry 148. It is through the antenna coil 150 that charging, forward telemetry and back telemetry signals may be received and sent to an external device, such as an external programmer or charging circuit, as described more fully in the above-referenced U.S. patent application, Ser. No. 09/626,010. (In practice, separate coils may be used for charging, forward telemetry and back telemetry functions, as described more fully in the above-referenced U.S. Pat. No. 6,516,227, but for purposes of the present invention those distinctions are not relevant.)

In FIG. 4, the antenna coil(s) 150 is shown as being outside the hermetically sealed case of the IPG. In such configuration, feed-through terminals 103 are used to allow the coil(s) to be electrically connected to the charging and telemetry circuitry 148, which are inside the hermetically sealed case. Alternatively, if the case is made from a non-ferromagnetic material, such as titanium, or ceramic, the coil(s) 150 may be located inside of the hermetically sealed case.

It is to be emphasized that that which is shown in FIG. 4 is intended to be functional, and not limiting. Those of skill in the art will be able to fashion appropriate circuitry, whether embodied in digital circuits, analog circuits, software and/or firmware, or combinations thereof, in order to accomplish the desired functions.

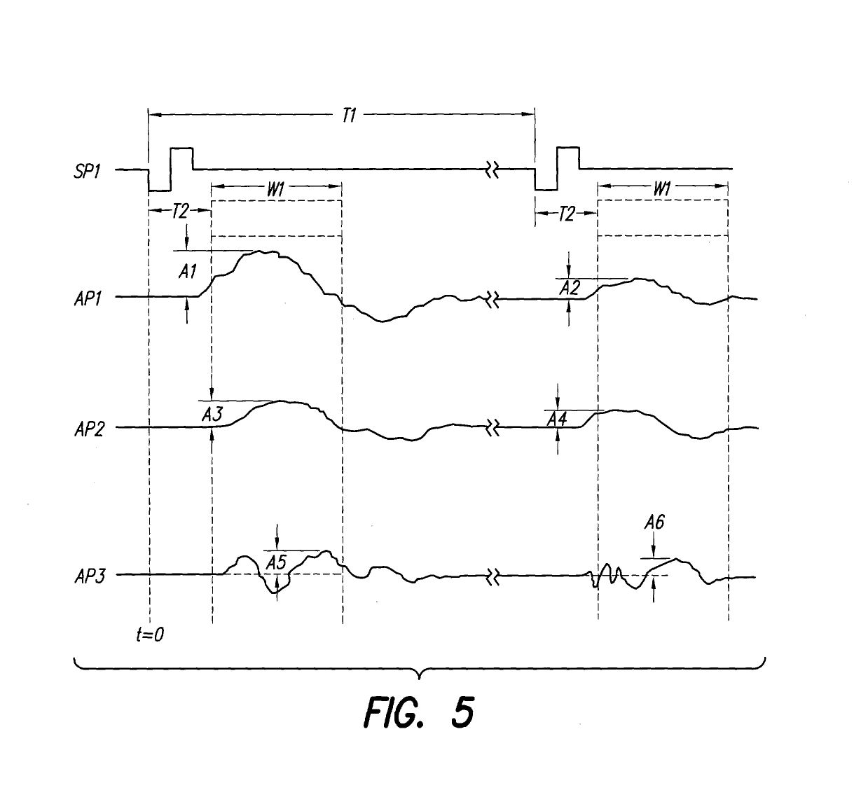

FIG. 5 is a timing waveform diagram that depicts various types of representative action potentials (AP1, AP2, AP3) that may be sensed as a function of time in response to a generated stimulus pulse (SP1). As seen in FIG. 5, a stimulation pulse SP1 is applied to selected electrodes at time t=0. Shortly after the pulse SP1 is applied, e.g., at a time T2 after t=0 (where T2 may be, e.g., from 0.1 to 5 milliseconds (msec)), a monitoring window, having a time duration of W1 msec, begins. The width of the monitoring time window W1 may range, e.g., from 1-10 msec. During the time window W1, the sense electrodes are monitored for the presence of an action potential.

One representative action potential signal, AP1, shown in FIG. 5 has an amplitude A1 during a first monitoring window W1. At a known time T1 thereafter, during a second monitoring window W1, the action potential AP1 has changed to an amplitude of A2, where A2 is markedly less than the amplitude A1. Here, a marked change may be defined as one that has changed greater than about 10-20% from a running average of the last 5-10 amplitude measurements. Such marked change in amplitude of the action potential indicates a significant change has occurred in the coupling efficiency. Such change could be caused, for example, by a postural change or relative movement between the electrode array and nerve tissue.

In accordance with the present invention, and in response to sensing such a marked change between the action potential amplitudes A1 and A2, correction circuitry programmed or wired into the μC 130 causes the amplitude of the stimulation current to increase, thereby maintaining the efficacy of the applied stimulus. Had the amplitude A2 been markedly greater than the amplitude A1, then the correction circuitry would cause the amplitude of the stimulation current to decrease.

Another representative action potential signal, AP2, shown in FIG. 5 has an amplitude A3 during the first monitoring window W1. At a known time T1 thereafter, during a second monitoring window W1, the action potential AP2 has changed to an amplitude of A4, where A4 is only significantly less than the amplitude A3. As used here, the term “slightly” may be defined as a difference of less than about 5% of the running average of the last 10-20 measurements as compared to the running average of 10-20 measurements taken several hours, e.g., 12-36 hours, earlier. Such slight change in amplitude of the action potential indicates a corresponding slight change has occurred in the coupling efficiency between the prior time and the present time. Such change could be caused, for example, by a scare tissue maturation.

In accordance with the present invention, and in response to sensing such a slight change between the action potential amplitudes A3 and A4, correction circuitry programmed or wired into the μC 130 causes the amplitude of the stimulation current to increase a small amount, thereby maintaining the efficacy of the applied stimulus at a constant level. Had the amplitude A4 been slightly greater than the amplitude A3, then the amplitude of the stimulation current would be decreased a small amount.

Yet another representative action potential signal, AP3, shown in FIG. 5 has an amplitude A5 during the first monitoring window W1. At a known time T1 thereafter, during a second monitoring window W1, the action potential AP3 has changed to an amplitude of A6, where A6 may or may not be much different than A5. However, the morphology of the AP3 waveform (where “morphology” means the waveform shape) has changed markedly. Such marked change in the morphology of the action potential waveform may likewise be used as an indicator that a change has occurred in the coupling efficiency.

Known techniques for determining the morphology of a waveform may be used to determine changes in the morphology over time. See, e.g., U.S. Pat. No. 5,685,315, incorporated herein by reference. A simple technique, for example, integrates the action potential waveform, or other physiologic waveform, during the monitoring window W1 so as to calculate the area under the morphology waveform curve. While such simple technique is not fool proof (because it is possible for many different shaped curves to have the same area under the curve over a fixed time), it is generally adequate for purposes of the present invention to detect a change in coupling efficiency.

In accordance with the present invention, and in response to sensing such a marked change between the morphology of the action potential or other physiologic waveforms, correction circuitry programmed or wired into the μC 130 causes the magnitude of the stimulation current to increase, thereby maintaining the efficacy of the applied stimulus at a desired level.

It is the energy content of the stimulus pulse that is adjusted in accordance with the invention when a change in the coupling efficiency has been detected. The energy content of the stimulus pulse is readily adjusted by adjusting the amplitude of the stimulus pulse. However, the energy content can also be adjusted by changing the width, or duration, of the stimulus pulse waveform, as well as the frequency with which the stimulus pulse is applied. Thus, as used herein, the term “amplitude”, or “magnitude”, when used to describe the characteristics of the applied stimulus pulse, is meant to include pulse amplitude or pulse width or pulse frequency, or combinations of amplitude, width and frequency.

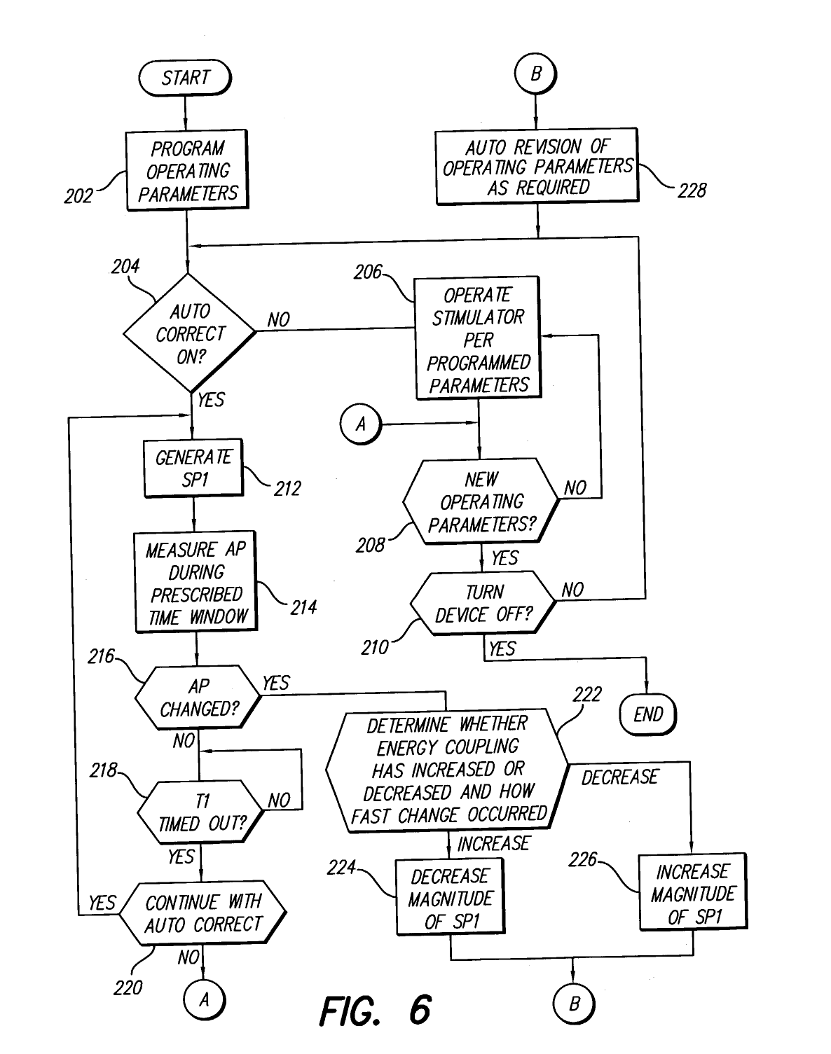

FIG. 6 is a flow chart that shows a method of practicing the invention in accordance with one embodiment thereof. Although the flow chart is directed to the measurement of the action potential, or AP, it is to be understood that other physiologic parameter(s) other than the AP could also be measured, as has been previously indicated. Thus, in the description of FIG. 6 that follows, it should be understood that whenever “AP” is stated or used, the measurement of other physiologic parameters, such as optical properties, pressure, chemistry, or the like, of the tissue near the electrodes, may be made in lieu of, or as a supplement to, the measurement of the AP.

As seen in FIG. 6, a first step of the method involves programming the operating parameters (block 202) into the IPG circuitry. Such operating parameters include not only the operating regime for the neural stimulation system, e.g., stimulation pulse amplitude, width, frequency, and electrodes, but also the parameters used by the invention to determine when a sufficient change in the sensed action potential has occurred so as to trigger the auto correction features of the invention.

Once all the operating parameters have been programmed, then a determination is made as to whether the auto correction feature of the invention has been programmed ON (block 204). If not, then the stimulator operates in accordance with its programmed operating regime without invoking the auto correction feature (block 206). Should new programming occur (block 208), such that new operating parameters are loaded into the device memory (which may include turning the auto correction feature ON), and should the device not be turned OFF (block 210), then the process continues by returning to block 204, where a new determination is made as to whether the auto correction feature is turned ON.

If auto correction is turned ON (block 204), then a stimulus pulse SP1 is generated and applied to the specified electrodes (block 212). Next, the action potential AP, or other physiologic parameter, is measured during the prescribed time window (block 214). Once the AP or other parameter has been measured, then a determination is made as to whether the AP or other parameter has changed (block 216). If not, then the process waits for the next sample time (block 218), and if auto correction is not turned off (block 220), then the process repeats (blocks 212, 214, 216).

If a determination is made that the action potential AP or other parameter has changed (block 216), then a determination is made as to whether the energy coupling has increased or decreased, and how fast the change has occurred (block 222). If the coupling has increased, then the magnitude of the stimulation pulse SP1 is decreased an appropriate amount (block 224). If the coupling has decreased, then the magnitude of the stimulation pulse SP1 is increased an appropriate amount (block 226). Then, other parameters are revised or adjusted, as appropriate (block 228) so that all the operating parameters are compatible with the changes made to SP1, and the process repeats, starting at block 204.

As described above, it is thus seen that the present invention provides a neural stimulation system wherein the output stimulus magnitude is automatically corrected or adjusted in order to compensate for coupling efficiency variations in the electrode-to-neural-tissue interface that cause more or less energy to reach the neural tissue from the electrode. Variations in the coupling efficiency are determined, in one preferred embodiment, by sensing changes in a sensed action potential. In other preferred embodiments, variations in coupling efficiency are determined by sensing changes in the optical properties of the tissue surrounding the target neural tissue that is stimulated, by sensing changes in the pressure present within the tissue surrounding the target neural tissue, by sensing changes in the chemical properties of fluids within the epidural space, or the like.

As further described above, it is seen that the invention provides a method of neural stimulation that includes measuring action potentials, or other suitable physiologic parameter(s), that are indicative of the coupling efficiency of the electrical stimulation current to the neural tissue, and automatically adjusting the magnitude of subsequent stimulating current pulses in order to compensate for variations in the measured coupling efficiency.

While the invention herein disclosed has been described by means of specific embodiments and applications thereof, numerous modifications and variations could be made thereto by those skilled in the art without departing from the scope of the invention set forth in the claims.

What is claimed is:

1. A method of neural stimulation comprising:
measuring the coupling efficiency of an electrical stimulation current applied to neural tissue; and
automatically adjusting the magnitude of subsequent stimulating current pulses in order to compensate for variations in the measured coupling efficiency.

2. The method of claim 1 wherein measuring the coupling efficiency comprises measuring the optical properties of tissue in close proximity to the neural tissue.

3. The method of claim 1 wherein measuring the coupling efficiency comprises measuring the pressure within the tissue in close proximity to the neural tissue;

4. The method of claim 1 wherein measuring the coupling efficiency comprises measuring the pH of fluids within the epidural space.

5. The method of claim 1 wherein measuring the coupling efficiency comprises measuring action potentials by
applying a current stimulus to a first electrode in close proximity to the neural tissue;
monitoring the first electrode for an action potential during a monitoring time window that begins a time T2 after application of the current stimulus, wherein the time window has a duration of W1 seconds; and
measuring the peak amplitude of the action potential during the time window W1.

6. The method of claim 5 wherein the time T2 comprises 0 to 5 milliseconds.

7. The method of claim 5 wherein the time window W1 has a duration of from 1 to 10 milliseconds.

8. The method of claim 1 wherein measuring coupling efficiency comprises measuring action potentials by:
applying a current stimulus to a first electrode in close proximity to the neural tissue;
monitoring the first electrode for an action potential during a monitoring time window that begins a time T2 after application of the current stimulus, wherein the time window has a duration of W1 seconds; and
integrating the action potential during the time window W1.

9. The method of claim 1 wherein measuring coupling efficiency comprises measuring action potentials by:
applying a current stimulus to a first stimulating electrode in close proximity to the neural tissue;
monitoring a second stimulating electrode adjacent the first stimulating electrode for an action potential during a monitoring time window that begins a time T2 after application of the current stimulus, wherein the time window has a duration of W1 seconds; and
integrating the action potential during the time window W1.

10. A neural stimulation system comprising:
a plurality of electrodes;
a pulse generator connected to the plurality of electrodes, wherein the pulse generator provides stimulation current pulses to selected electrodes of the plurality of electrodes at a prescribed magnitude;
means for measuring a physiologic parameter indicative of coupling efficiency;
means for determining a change in the physiologic parameter over time; and
auto correction means for adjusting the magnitude of the stimulation current pulse as a function of the amount of change detected in the physiologic parameter over time.

11. The neural stimulation system of claim 10 wherein the plurality of electrodes are adapted to be inserted into a patient's epidural space, and wherein the means for measuring a physiologic parameter comprises means for measuring chemical properties of fluids and tissue in the epidural space.

12. The neural stimulation system of claim 10 wherein the plurality of electrodes are adapted to be inserted into a patient's epidural space, and wherein the means for measuring a physiologic parameter comprises means for measuring pressure that occurs within the epidural space.

13. The neural stimulation system of claim 10 wherein the plurality of electrodes are adapted to be inserted in close proximity to target neural tissue of a patient, and wherein the means for measuring a physiologic parameter comprises means for measuring optical properties of tissue and fluids adjacent the target neural tissue.

14. An implantable spinal cord stimulation system comprising:
means for measuring the coupling efficiency of an electrical stimulation current applied to neural tissue in or near the epidural space; and
means for automatically adjusting the magnitude of subsequent stimulating current pulses in order to compensate for variations in the measured coupling efficiency.

15. The implantable spinal cord stimulation system of claim 14 wherein the means for measuring the coupling efficiency comprises means for measuring the optical properties of tissue in close proximity to the neural tissue.

16. The implantable spinal cord stimulation system of claim 14 wherein the means for measuring the coupling efficiency comprises means for measuring pressure within the tissue and fluids in, or in close proximity to, the epidural space.

17. The implantable spinal cord stimulation system of claim 14 wherein the means for measuring the coupling efficiency comprises means for measuring chemical properties of tissue or fluids within the epidural space.
